(19) United States
(12) Patent Application Publication
(10) Pub. No.: US 2014/0205654 A1
(43) Pub. Date: Jul. 24, 2014

(54) LIPOSOMAL VANCOMYCIN FOR THE TREATMENT OF MRSA INFECTIONS

(71) Applicants: George Y. Liu, Los Angeles, CA (US); Abdel Omri, Sudbury (CA)

(72) Inventors: George Y. Liu, Los Angeles, CA (US); Abdel Omri, Sudbury (CA)

(73) Assignee: CEDARS-SINAI MEDICAL CENTER, Los Angeles, CA (US)

(21) Appl. No.: 14/113,793

(22) PCT Filed: Apr. 26, 2012
(86) PCT No.: PCT/US12/35134

§ 371 Date: Dec. 10, 2013

Related U.S. Application Data
(60) Provisional application No. 61/479,305, filed on Apr. 26, 2011.

(51) Int. Cl. A61K 9/127; A61K 38/14

(52) U.S. Cl. CPC A61K 9/127; A61K 38/14
USPC 424/450; 514/2.7

(57) ABSTRACT

The present invention discloses formulations of liposomal vancomycin that are highly effective in the treatment of bacterial infections, and particularly MRSA infections. The present invention further teaches methods of using the formulations disclosed herein for the treatment of bacterial infections. The inventors determined that certain formulations disclosed herein are likely to result in lower toxicity than what is normally associated with vancomycin treatment.

### CROSS-REFERENCE TO RELATED APPLICATIONS

This application claims priority from U.S. Provisional Patent Application No. 61/479,305 filed on Apr. 26, 2011, which is incorporated herein by reference in its entirety.

### FIELD OF THE INVENTION

The invention disclosed herein generally relates to the treatment of bacterial infections.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Methicillin-resistant S. aureus poses a major problem to public health worldwide. MRSA strains with increased resistance to vancomycin cause infections that are associated with greater morbidity and threaten the use of this once gold standard anti-staphylococcal drug. There is a need in the art for improved treatments for bacterial infections, and particularly for those infections that are resistant to traditional antibiotics.

### SUMMARY OF THE INVENTION

In some embodiments, the invention teaches a composition including vancomycin, a DCP liposome and/or a DMPG liposome. In some embodiments, the composition includes vancomycin, DSPC, DCP and cholesterol. In some embodiments, the DSPC, DCP and cholesterol are in a molar ratio of 7:2:1. In some embodiments, the composition includes vancomycin, DSPC, DMPG and cholesterol. In certain embodiments, the DSPC, DMPG and cholesterol are in a molar ratio of 7:2:1.

In certain embodiments, the invention teaches a method of treating an infection in an individual, including: providing a composition including vancomycin, a DCP liposome and/or a DMPG liposome; and administering a therapeutically effective amount of the composition to the individual so as to treat the infection.

In some embodiments, the composition includes vancomycin, DSPC, DCP and cholesterol. In some embodiments, the DSPC, DCP and cholesterol are in a molar ratio of 7:2:1. In some embodiments, the composition includes DSPC, DMPG and cholesterol. In some embodiments, the DSPC, DMPG and cholesterol are in a molar ratio of 7:2:1. In some embodiments, the infection is a bacterial infection caused by a microorganism selected from the group consisting of: Staphylococcus aureus (S. aureus), methicillin-resistant S. aureus, Vancomycin-Intermediate S. aureus, S. pneumoniae, E. faecalis, E. faecium, Coagulase-negative staphylococci and combinations thereof. In some embodiments, the infection is caused by methicillin-resistant S. aureus.

In certain embodiments, the invention teaches a kit for treating a bacterial infection, including: a composition including vancomycin, a DCP liposome and/or a DMPG liposome; and instructions for the use thereof to treat a bacterial infection in an individual. In some embodiments, the composition includes vancomycin, DSPC, DCP and cholesterol. In some embodiments, the DSPC, DCP and cholesterol are in a molar ratio of 7:2:1. In some embodiments, the composition includes vancomycin, DSPC, DMPG and cholesterol. In some embodiments, the DSPC, DMPG and cholesterol are in a molar ratio of 7:2:1. In some embodiments, the bacterial infection is caused by a microorganism selected from the group consisting of: Staphylococcus aureus (S. aureus), methicillin-resistant S. aureus, Vancomycin-Intermediate S. aureus, S. pneumoniae, E. faecalis, E. faecium, Coagulase-negative staphylococci and combinations thereof. In some embodiments, the infection is caused by methicillin-resistant S. aureus.

### BRIEF DESCRIPTION OF THE DRAWINGS

Exemplary embodiments are illustrated in referenced figures. It is intended that the embodiments and figures disclosed herein are to be considered illustrative rather than restrictive.

FIG. 1 demonstrates, in accordance with an embodiment of the invention, a time-kill assay of MRSA. LAC or NRS-35 was incubated with free or DCP liposomal vancomycin. Shown are surviving CFUs at select time points. (a) LAC: For the 3 hr time point, p values for free versus DCP VAN groups were <0.005 for concentrations 0.3 mg/L and 1.25 mg/L, and <0.01 for concentration 0.6 mg/L. For the 6 hr time point, the p value was 0.02 for free versus DCP VAN at 0.6 mg/L. (b) NRS-35: p values for free versus DCP VAN groups were <0.005 (3 hr) and <0.05 (6 hr) for concentrations 1.25 mg/L. The results are representative of three experiments.

FIG. 2 demonstrates, in accordance with an embodiment of the invention, efficacy of free and DCP liposomal vancomcyin in the treatment of MRSA systemic infection. CD1 mice were infected intraperitoneally with 4×10⁶ CFUs of MRSA (LAC). Mice were treated one hour later with PBS, DCP liposomal vancomycin, or free vancomycin. A. Spleen bacterial burden at 24 hours. p values were 0.001 for PBS versus DCP VAN, and 0.07 for DCP VAN versus free VAN. B. Kidney bacterial burden after 24 hours. p values were <0.001 for PBS versus DCP VAN, 0.03 for PBS versus free VAN, and 0.015 for DCP VAN versus free VAN. Lines represent median values.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3rd ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

As used herein:

The acronym “MRSA” means methicillin-resistant Staphylococcus aureus (S. aureus).

The acronym “MIC” means minimum inhibitory concentration.

The acronym “MBC” means minimum bactericidal concentration.

The acronym “DSPC” means 1,2-distearoyl-sn-glycero-3-phosphocholine.

The acronym “DMPG” means dimyristoylphosphatidylglycerol.

The acronym “DCP” means dicetyl phosphate.

The abbreviation “CHOL” means cholesterol.

As used herein, “beneficial results” may include, but are in no way limited to, lessening or alleviating the severity of the disease condition, preventing the disease condition from worsening, curing the disease condition, preventing the disease condition from developing, lowering the chances of a subject developing the disease condition and prolonging a subject's life or life expectancy.

“Conditions” and “disease conditions,” as used herein may include, but are in no way limited to bacterial infection, conditions associated therewith and combinations thereof.

“Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

“Treatment” and “treating,” as used herein refer to both therapeutic treatment and prophylactic or preventative measures, wherein the object is to slow down (lessen) the targeted pathologic condition, prevent the pathologic condition, pursue or obtain beneficial results, or lower the chances of the individual developing the condition even if the treatment is ultimately unsuccessful. Those in need of treatment include those already with the condition as well as those prone to have the condition or those in whom the condition is to be prevented.

In some embodiments, the numbers expressing quantities of ingredients, properties such as molecular weight, reaction conditions, and so forth, used to describe and claim certain embodiments of the application are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Methicillin-Resistant Staphylococcus aureus (MRSA) infections are among the most challenging infections that plague public health. Over the past decade, the emergence of Community-Associated MRSA (CA-MRSA), particularly USA300, has contributed significantly to the increased burden of infections¹⁻². The gold standard for treatment of MRSA is vancomycin, a large glycopeptide antibiotic that inhibits peptidoglycan biosynthesis on the surface of bacteria. However, many clinical studies have shown that S. aureus strains with vancomycin MIC above 1.5 are associated with greater morbidity and treatment failure as they may render MRSA harder to kill within specific host tissues.

A strategy to enhance antimicrobial efficacy of antimicrobial reagents is the encapsulation of the reagents within liposomes to enhance the delivery of drugs to certain host tissues³. Liposomes have been shown to improve antibiotic killing of bacteria engulfed by phagocytic cells, but depending on their composition they could also enhance the delivery of antibiotics to the bacterial cytoplasm to directly increase their antimicrobial efficacy⁴. Additionally, liposome entrapment frequently reduces toxicity associated with drugs, and therefore has been used as a method to permit higher drug dosing. For targeting of S. aureus, liposomal encapsulation of piperacillin has been shown to improve growth inhibition of S. aureus⁵. In previous studies, entrapment of vancomycin within different preparations of liposomes augmented the elimination of macrophage-engulfed MRSA⁶⁻⁷, but largely did not improve killing of extracellular S. aureus in vitro⁷. As disclosed herein, the inventors demonstrate enhanced anti-staphylococcal activity of two liposomal formulations of vancomycin against free MRSA.

In some embodiments, the invention teaches a composition of liposomal vancomycin including vancomycin, DCP liposomes and/or DMPG liposomes. In some embodiments, the liposomal vancomycin includes vancomycin, DSPC, DCP and cholesterol. In some embodiments, the DSPC, DCP and cholesterol are in a molar ratio of 7:2:1. In other embodiments, the liposomal vancomycin includes vancomycin, DSPC, DMPG and cholesterol. In some embodiments, the DSPC, DMPG and cholesterol are in a molar ratio of 7:2:1.

In some embodiments, the invention teaches a method of treating an infection in an individual, including providing a composition including vancomycin and a DCP liposome and/or a DMPG liposome; and administering a therapeutic dose of the composition to the individual. In some embodiments, the liposomal vancomycin includes vancomycin, DSPC, DCP and cholesterol. In some embodiments, the DSPC, DCP and cholesterol are in a molar ratio of 7:2:1. In some embodiments, the liposomal vancomycin includes vancomycin, DSPC, DMPG and cholesterol. In some embodiments, the DSPC, DMPG and cholesterol are in a molar ratio of 7:2:1. In some embodiments, the infection treated by one or more of the liposomal vancomycin formulations disclosed herein is an infection caused by a microorganism selected from the group consisting of: Staphylococcus aureus (S. aureus), methicillin-resistant S. aureus, Vancomycin-Intermediate S. aureus, S. pneumoniae, E. faecalis, E. faecium, Coagulase-negative staphylococci, and combinations thereof. In some embodiments, the infection is caused by methicillin-resistant S. aureus. In some embodiments, the infection is caused by Gram-positive bacteria. In some embodiments, the individual treated is a mammal. In some embodiments, the individual treated is a human. In certain embodiments, the individual treated is immune compromised. In some embodiments, the individual treated has one or more secondary infections caused by one or more additional pathogens.

The pharmaceutical compositions according to the methods and kits of the invention may be formulated for delivery via any route of administration. “Route of administration” may refer to any administration pathway known in the art, including but not limited to aerosol, nasal, oral, transmucosal, transdermal or parenteral. “Transdermal” administration may be accomplished using a topical cream or ointment or by means of a transdermal patch. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection. Via the enteral route, the pharmaceutical compositions can be in the form of tablets, gel capsules, sugar-coated tablets, syrups, suspensions, solutions and the like. Via the topical route, the pharmaceutical compositions based on compounds according to the invention may be formulated for treating the skin and mucous membranes and are in the form of ointments, creams, milks, salves, powders, impregnated pads, solutions, gels, sprays, lotions or suspensions. Via the ocular route, they may be in the form of eye drops.

The pharmaceutical compositions according to the methods and kits of the invention can also contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the methods and kits of the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of syrup, an elixir, an emulsion or an aqueous or non-aqueous suspension. Such a liquid formulation may be administered directly p.o. or filled into a soft gelatin capsule.

The pharmaceutical compositions according to the methods and kits of the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

Typical dosages can be in the ranges recommended by the manufacturer where known therapeutic compounds are used, and also as indicated to the skilled artisan by the in vitro responses or responses in animal models. Such dosages typically can be reduced by up to about one order of magnitude in concentration or amount without losing the relevant biological activity. Thus, the actual dosage will depend upon the judgment of the physician, the condition of the patient, and the effectiveness of the therapeutic method based, for example, on in vitro responsiveness or the responses observed in the appropriate animal models.

In some embodiments, the present invention is also directed to a kit to treat bacterial infections. In some embodiments, the kit is useful for treating MRSA infections in an individual. The kit is an assemblage of materials or components, including one or more of the compositions described herein. Thus, in some embodiments the kit contains a composition including vancomycin, DCP liposomes and/or DMPG liposomes. In some embodiments, the liposomal vancomycin includes vancomycin, DSPC, DCP and cholesterol. In some embodiments, the DSPC, DCP and cholesterol are in a molar ratio of 7:2:1. In other embodiments, the liposomal vancomycin includes vancomycin, DSPC, DMPG and cholesterol. In some embodiments, the DSPC, DMPG and cholesterol are in a molar ratio of 7:2:1.

The exact nature of the components configured in the inventive kit depends on its intended purpose. For example, some embodiments are configured for the purpose of treating bacterial infections. In some embodiments, the bacterial infection is caused by a microorganism selected from the group consisting of: Staphylococcus aureus (S. aureus), methicillin-resistant S. aureus, Vancomycin-Intermediate S. aureus, S. pneumoniae, E. faecalis, E. faecium, Coagulase-negative staphylococci and combinations thereof. In an embodiment, the infection is caused by methicillin resistant S. aureus. In some embodiments, the kit is configured particularly for the purpose of treating mammalian subjects. In other embodiments, the kit is configured particularly for the purpose of treating human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to effect a desired outcome, such as to treat a bacterial infection. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as the inventive compositions disclosed herein and the like. The packaging material is constructed by well-known methods, preferably to provide a sterile, contaminant-free environment. The packaging materials employed in the kit are those customarily utilized in treating bacterial infections. As used herein, the term “package” refers to a suitable solid matrix or material such as glass, plastic, paper, foil, and the like, capable of holding the individual kit components. Thus, for example, a package can be a glass vial used to contain suitable quantities of an inventive composition used to treat a bacterial infection. The packaging material generally has an external label which indicates the contents and/or purpose of the kit and/or its components.

The following non-limiting examples are provided to further illustrate embodiments of the invention disclosed herein. It should be appreciated by those of skill in the art that the techniques disclosed in the examples that follow represent approaches that have been found to function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### EXAMPLES

### Example 1

### Bacterial Strains, Mice, and Chemical Products

The MRSA strains used in the study were: NRS-35 (a Hospital-Associated MRSA from NARSA), LAC (a CA-MRSA strain of the pulsotype USA 300), ATCC 29213, ATCC 433000, and six additional MRSA isolates from the Cedars-Sinai clinical laboratory (Cedars-Sinai IRB exemption Pro0022541). Ten-week old CD1 male mice were purchased from Charles River. 1,2-Distearoyl-sn-glycero-3-phosphocholine (DSPC) and cholesterol were obtained from Northern Lipids Inc. (Burnaby, BC, Canada). Dicethylphosphate (DCP) and dimyristoylphoshatidylglycerol (DMPG were purchased from Sigma-Aldrich (St. Louis, Mo., USA). Vancomycin was procured from Gold Biotechnology (St. Louis, Mo., USA).

### Example 2

### Liposome Preparation

A lipid mixture of either DSPC:DMPG:CHOL in a 7:2:1 molar ratio (DSPC 71.5 mg, DMPG 17.8 mg, and cholesterol 5 mg) or DSPC:DCP:CHOL in a 7:2:1 molar ratio (DSPC 71.5 mg, DCP 14.1 mg, and cholesterol 5 mg) were dissolved in 1 mL of chloroform/methanol (2:1 v/v ratio) in a round bottom Erlenmeyer flask. The organic solvents were removed under vacuum at 55° C. using a rotary evaporator (Buchi-Rotavapor R205, Brinkmann, Toronto, ON, Canada). This process produced a thin dried lipid film.

A 1 ml aqueous solution of vancomycin (50 mg/mL) was added to the thin dry lipid film and hand shaken in a 55° C. water bath for 1 minute. The lipid suspension was then sonicated for 5 minutes (Sonic Dismembrator Model 500, Fischer Scientific, USA) while submerged in an ice-bath. The sonicator was not in direct contact with the liposome suspension at any time. The suspension was freeze-dried overnight for preservation (Labconco model 77540, USA).

The mean diameters of liposomes were determined by photon correlation spectroscopy using a NICOMP 270 Submicron Particle Size Analyzer (Santa Barbara, Calif., USA) operating at 23° C. The sizes for the liposomes were 527.6 nm±58.2 nm. Vancomycin at 50 mg/ml was used for the preparation of liposomal vancomyin. The encapsulation efficiency of these liposomes, defined as the ratio of the amount of active compound remaining inside the liposomes to the initial amount of vancomycin introduced, was determined by radial diffusion from wells cut in Mueller-Hinton agar (Becton Dickinson) with a lawn of S. aureus ATCC 29213, and the vancomycin concentration calculated from a standard curve. The efficiency was 9% for DCP and 20% for DMPG. For rehydration of the liposomes, Phosphate Buffer Solution (PBS) was added, vortexed and heated in water bath at 44° C. for 30 minutes, and then centrifuged at 12000 rpm for 20 minutes. The supernatant was discarded, and the pellet was reconstituted with PBS, and used within two days.

### Example 3

### MRSA Killing Kinetic Assay

MRSA strains were grown overnight at 37° C. with shaking in Todd Hewitt Broth (THB). The next day, the bacteria were washed twice in PBS, added to THB with or without antibiotics to achieve a final concentration of approximately 2×10⁵ CFUs/ml, and then incubated at 37° C. with shaking. At 0, 3, and 6 hours, the bacteria were plated on agar for enumeration of surviving CFUs.

### Example 4

### Treatment of Systemic MRSA Infection

Overnight MRSA culture was diluted in THB, grown to an OD₆₀₀ of 0.6 at 37° C. with shaking, centrifuged, and then washed twice in PBS. Ten week-old CD-1 male mice were infected intraperitoneally with 3.9×10⁶ CFUs of MRSA. One hour after infection, the mice were injected intraperitoneally with PBS, 50 mg/kg of DCP liposomal vancomycin, or 50 mg/kg of free vancomycin. After 24 hours the mice were sacrificed, and spleen and kidneys were harvested, homogenized, and plated on agar for enumeration of CFUs. The animal infection experiment was approved by the Cedars-Sinai Committee on the Use and Care of Animals and performed using accepted veterinary standards.

### Example 5

### Statistical Analysis

For the time-kill assays, comparisons were made using unpaired Student's t-test. For the murine study, analysis of variance (ANOVA) for the means of the groups was calculated, and p values<0.05 were considered statistically significant.

### Example 6

### MICs and MBCs of Liposomal Vancomycin Formulations

Two liposomal vancomycin formulations were evaluated in the study, designated DCP and DMPG based on their inclusion in the respective liposomal preparations. As shown in Table 1, both DCP and DMPG vancomycin showed 2 to 4 fold lower MICs compared to free vancomycin against the HA-MRSA strain NRS-35 and CA-MRSA strain LAC. MBCs were approximately 4 fold lower for the liposomal antibiotics compared to free vancomycin (Table 2). DCP vancomycin was further evaluated against a panel of 7 MRSA strains and showed an approximately 2 fold lower MIC compared to free vancomycin (Table 1). Control liposome without antibiotics did not inhibit growth of the MRSA strains (data not shown).

### Example 7

### Efficacy of Liposomal Vancomycin in Clearance of MRSA

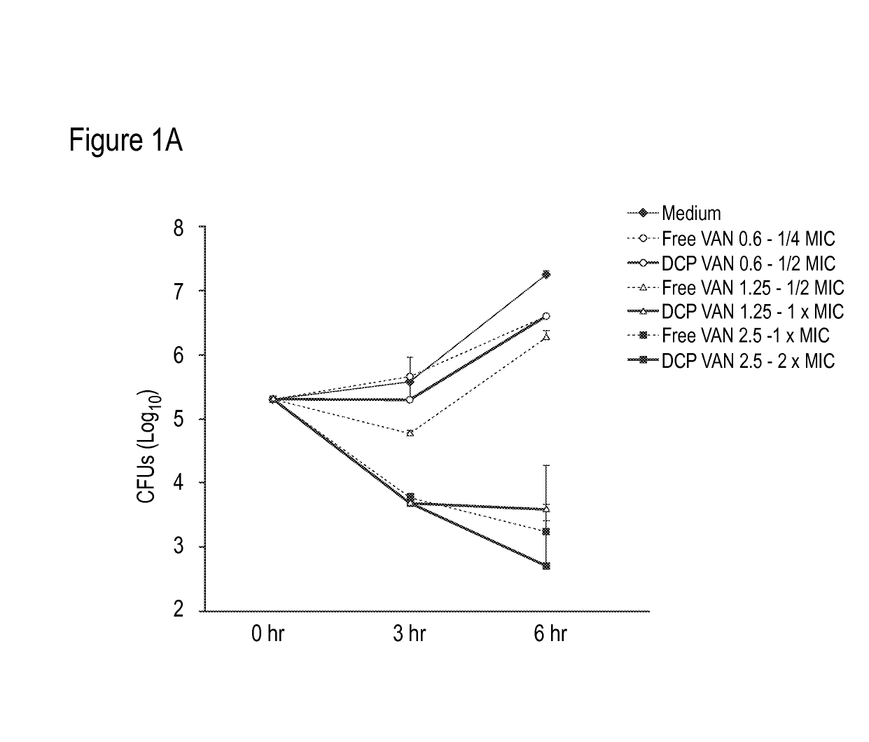

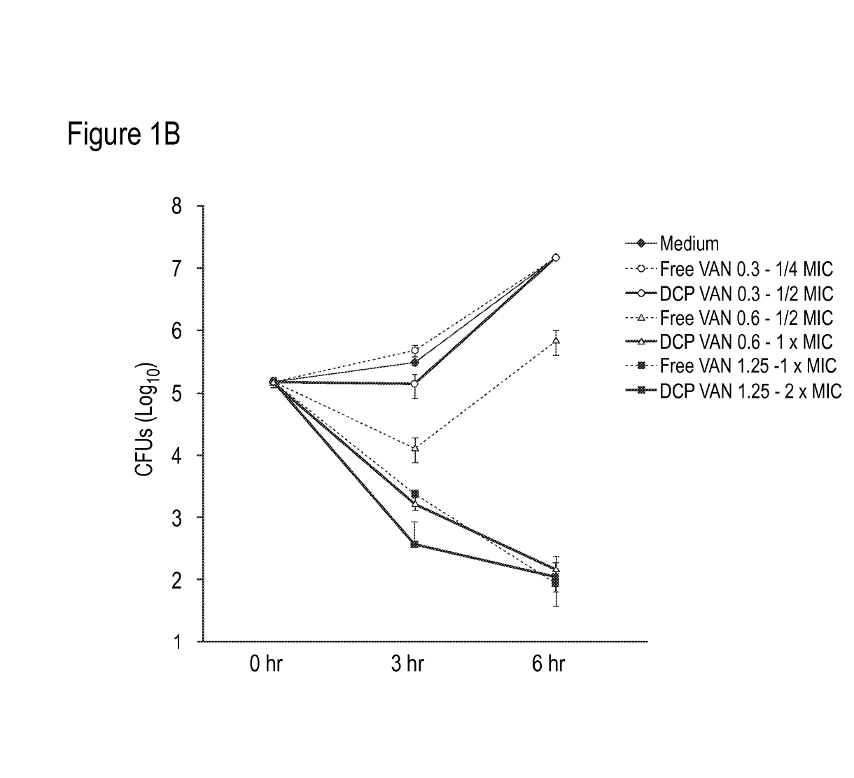

Next the antimicrobial activity of the DCP vancomycin was investigated in a time-kill assay. The concentrations chosen for free and liposomal vancomycin included ½×MIC, 1×MIC, and 2×MIC. As shown in FIG. 1a, DCP vancomycin demonstrated enhanced killing of MRSA (LAC) compared to regular vancomycin at all 3 concentrations after 3 hours. The difference between DCP and free vancomcin groups remained significant for the dose of 0.6 mg/L at 6 hour, but not for the other two doses. At a dose of 1.25 mg/L, DCP vancomycin also killed NRS-35 more effectively than free vancomycin both at 3 h and 6 h (FIG. 1b). No significant difference in killing was noted at other drug concentrations.

### Example 8

### Efficacy of DCP Liposomal Vancomycin in a Systemic MRSA Infection Model

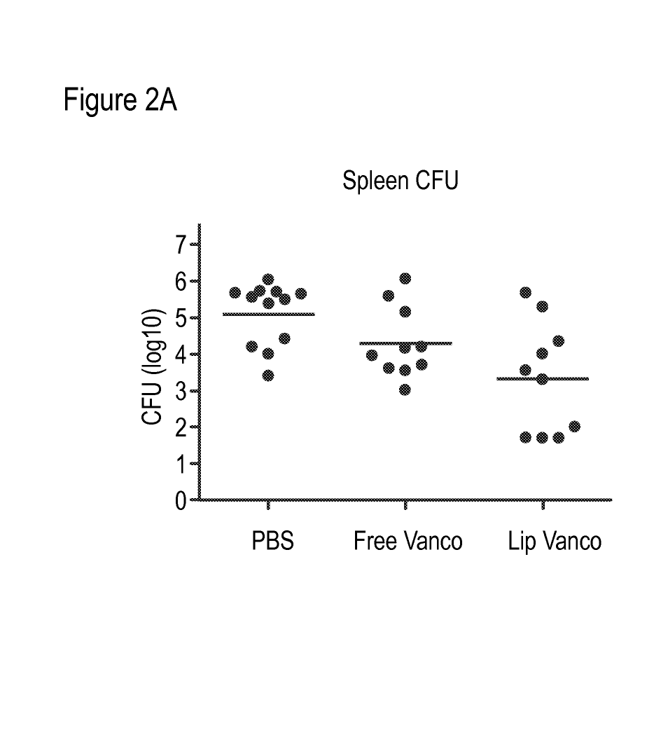

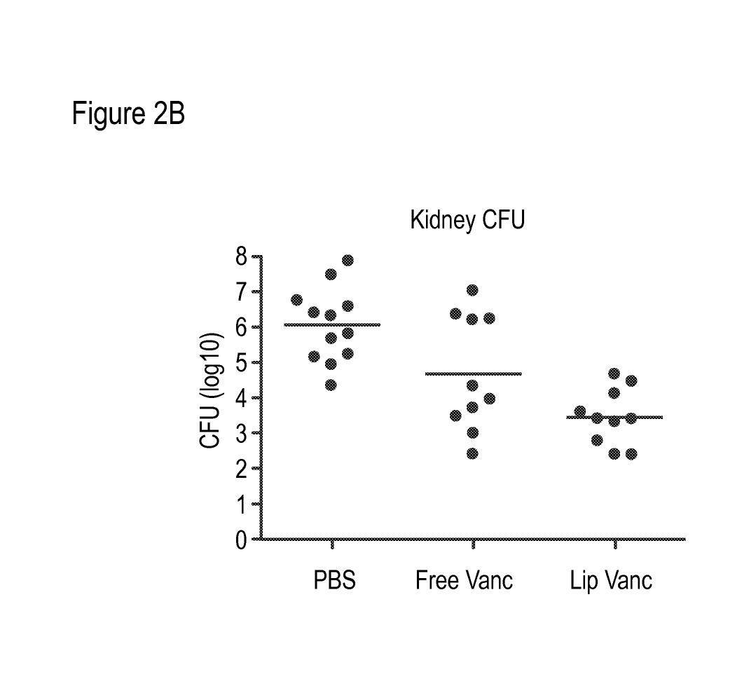

To evaluate the efficacy of DCP in vivo, groups of 10-12 mice were inoculated with MRSA (LAC) intraperitoneally. After an hour, the mice were administered PBS, 50 mg/kg of free vancomycin, or 50 mg/kg of DCP vancomycin. At 24 hour post infection, the CFU burden in the kidneys and spleen was enumerated (FIG. 2). Overall, DCP vancomycin reduced the bacterial burden by 2 to 3 logs in the spleen and kidneys compared to the PBS group (p<=0.001). Compared to free vancomycin treatment, DCP vancomycin treatment reduced the bacterial load in both kidneys and spleen by approximately one log. This was significant for the kidney CFUs, but not for the spleen CFUs.

### Example 9

### Determinations of MICs and MBCs

The MICs of free and liposomal antibiotics for all strains were determined by the broth dilution technique. Briefly, serial dilutions of free- or liposome-encapsulated antibiotic from 20 to 0.3 μg/ml were prepared. For bacterial suspensions, frozen stocks of MRSA were streaked on sheep blood agar plates. Single colonies were inoculated into fresh media and shaken for 20 hr at 37° C. The overnight bacteria were diluted and added to 96-well plates (Becton Dickinson) to achieve an approximate concentration of 1×10⁵. The inocula were confirmed by plating on agar. The 96-well plates were then incubated with or without shaking at 37° C., and the lowest concentration of antibiotic formulations that inhibited visible growth of bacteria was determined after 24 hours (MIC). Incubation of the plates with shaking (at 200 rpm) increased the consistency of MIC measurement for the liposomal preparations. For determination of MBC, the broths used for MIC determination were plated on agar, and the first dilution at which 99.9% of the original inocula were killed was identified. All MIC and MBC assays were performed at least three times.

### Example 10

### Tables

TABLE 1
Vancomycin MICs of MRSA strains

| | Free | DCP Liposomal | DMPG Liposomal |
|---|---|---|---|
| Bacterial | Vancomycin | Vancomycin | Vancomycin |
| Strain | (mg/L) | (mg/L) | (mg/L) |
| NRS-35 | 2.5 | 0.6-1.25 | 1.25 |
| LAC | 1.25-2.5 | 0.3-0.6 | 0.6 |
| ATCC 43300 | 1.25-2.5 | 1.25 | DNT |
| MRSA 78 | 1.25 | 0.6-0.3 | DNT |
| MRSA 79 | 1.25 | 0.6-1.25 | DNT |
| MRSA 80 | 1.25 | 0.6 | DNT |
| MRSA 81 | 1.25 | 0.6 | DNT |
| MRSA 82 | 2.5 | 1.25 | DNT |
| MRSA 83 | 1.25 | 0.6 | DNT |

DNT—did not test.

TABLE 2
Vancomycin MBCs of MRSA strains

| | Free | DCP Liposomal | DMPG Liposomal |
|---|---|---|---|
| Bacterial | Vancomycin | Vancomycin | Vancomycin |
| Strain | (mg/L) | (mg/L) | (mg/L) |
| NRS-35 | 2.5-5 | 0.6-1.25 | 0.6-1.25 |
| LAC | 2.5 | 0.6 | 0.6 |

The various methods and techniques described above provide a number of ways to carry out the invention. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Preferred embodiments of this application are described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are

not limited to that precisely as shown and described.

### REFERENCES

1. Stryjewski, M. E. & Chambers, H. F. (2008). Skin and soft-tissue infections caused by community-acquired methicillin-resistant Staphylococcus aureus. Clin Infect Dis 46 Suppl 5, S368-377.
2. Moran, G. J., Krishnadasan, A., Gorwitz, R. J., Fosheim, G. E., McDougal, L. K., Carey, R. B., et al. (2006). Methicillin-resistant S. aureus infections among patients in the emergency department. N Engl J Med 355, 666-674.
3. Torchilin, V. P. (2005). Recent advances with liposomes as pharmaceutical carriers. Nat Rev Drug Discov 4, 145-160.
4. Mugabe, C., Azghani, A. O. & Omri, A. (2006). Preparation and characterization of dehydration-rehydration vesicles loaded with aminoglycoside and macrolide antibiotics. Int J Pharm 307, 244-250.
5. Nacucchio, M. C., Bellora, M. J., Sordelli, D. O. & D'Aquino, M. (1985). Enhanced liposome-mediated activity of piperacillin against staphylococci. Antimicrob Agents Chemother 27, 137-139.
6. Pumerantz, A., Muppidi, K., Agnihotri, S., Guerra, C., Venketaraman, V., Wang, J., et al. (2011). Preparation of liposomal vancomycin and intracellular killing of meticillin-resistant Staphylococcus aureus (MRSA). Int J Antimicrob Agents 37, 140-144.
7. Onyeji, C. O., Nightingale, C. H. & Marangos, M. N. (1994). Enhanced killing of methicillin-resistant Staphylococcus aureus in human macrophages by liposome-entrapped vancomycin and teicoplanin. Infection 22, 338-342.
8. Kadry, A. A., Al-Suwayeh, S. A., Abd-Allah, A. R. & Bayomi, M. A. (2004). Treatment of experimental osteomyelitis by liposomal antibiotics. J Antimicrob Chemother 54, 1103-1108.
9. Anderson, M. & Omri, A. (2004). The effect of different lipid components on the in vitro stability and release kinetics of liposome formulations. Drug Deliv 11, 33-39.
10. Mugabe, C., Halwani, M., Azghani, A. O., Lafrenie, R. M. & Omri, A. (2006). Mechanism of enhanced activity of liposome-entrapped aminoglycosides against resistant strains of Pseudomonas aeruginosa. Antimicrob Agents Chemother 50, 2016-2022.

What is claimed is:

1. A composition comprising vancomycin, a DCP liposome and/or a DMPG liposome.

2. The composition of claim 1 comprising vancomycin, DSPC, DCP and cholesterol.

3. The composition of claim 2, wherein the DSPC, DCP and cholesterol are in a molar ratio of 7:2:1.

4. The composition of claim 1 comprising vancomycin, DSPC, DMPG and cholesterol.

5. The composition of claim 4, wherein the DSPC, DMPG and cholesterol are in a molar ratio of 7:2:1.

6. A method of treating an infection in an individual, comprising:
providing a composition comprising vancomycin, a DCP liposome and/or a DMPG liposome; and
administering a therapeutically effective amount of the composition to the individual so as to treat the infection.

7. The method of claim 6, wherein the composition comprises vancomycin, DSPC, DCP and cholesterol.

8. The method of claim 7, wherein the DSPC, DCP and cholesterol are in a molar ratio of 7:2:1.

9. The method of claim 6, wherein the composition comprises DSPC, DMPG and cholesterol.

10. The method of claim 9, wherein the DSPC, DMPG and cholesterol are in a molar ratio of 7:2:1.

11. The method of claim 6, wherein the infection is a bacterial infection caused by a microorganism selected from the group consisting of: Staphylococcus aureus (S. aureus), methicillin-resistant S. aureus, Vancomycin-Intermediate S. aureus, S. pneumoniae, E. faecalis, E. faecium, Coagulase-negative staphylococci and combinations thereof.

12. The method of claim 6, wherein the infection is caused by methicillin-resistant S. aureus.

13. A kit for treating a bacterial infection, comprising:
a composition comprising vancomycin, a DCP liposome and/or a DMPG liposome; and
instructions for the use thereof to treat a bacterial infection in an individual.

14. The kit of claim 13, wherein the composition comprises vancomycin, DSPC, DCP and cholesterol.

15. The kit of claim 14, wherein the DSPC, DCP and cholesterol are in a molar ratio of 7:2:1.

16. The kit of claim 13, wherein the composition comprises vancomycin, DSPC, DMPG and cholesterol.

17. The kit of claim 16, wherein the DSPC, DMPG and cholesterol are in a molar ratio of 7:2:1.

18. The kit of claim 13, wherein the bacterial infection is caused by a microorganism selected from the group consisting of: Staphylococcus aureus (S. aureus), methicillin-resistant S. aureus, Vancomycin-Intermediate S. aureus, S. pneumoniae, E. faecalis, E. faecium, Coagulase-negative staphylococci and combinations thereof.

19. The kit of claim 13, wherein the infection is caused by methicillin-resistant S. aureus.
